(19) United States
(12) Patent Application Publication
(10) Pub. No.: US 2021/0345854 A1
(43) Pub. Date: Nov. 11, 2021

(54) APPARATUS FOR CLEANING FOOTWEAR

(71) Applicant: Bright Kingdom Global Sourcing Limited, Kowloon (HK)

(72) Inventor: Harold W. WELLS, Huntington, NY (US)

(73) Assignee: Bright Kingdom Global Sourcing Limited, Kowloon (HK)

(21) Appl. No.: 17/313,666

(22) Filed: May 6, 2021

Related U.S. Application Data
(60) Provisional application No. 63/020,791, filed on May 6, 2020.

(51) Int. Cl. A47L 23/02; A47L 23/22; A46B 11/06; A46B 13/04; A47L 23/26; A61L 2/26; A61L 2/18

(52) U.S. Cl. CPC A47L 23/02; A47L 23/22; A46B 11/066; A46B 13/04; A61L 2202/15; A61L 2/26; A61L 2/18; A46B 2200/306; A61L 2202/17; A47L 23/263

(57) ABSTRACT

An apparatus for cleaning, sanitizing and/or disinfecting footwear. The apparatus comprises a set of brushes defining a footwear receiving space. A plurality of water carrying channels are provided for directing water into the footwear receiving space through said set of brushes. A soiled water receiving tank is provided for collecting soiled water from the footwear receiving space. The apparatus is arranged such that relative movement of a piece of footwear received in the footwear receiving space with respect to the set of brushes in the presence of water being directed into the footwear receiving space via the plurality of water carrying channels cleans at least one surface of the received piece of footwear.

This nonprovisional application claims priority to U.S. Provisional Application No. 63/020,791, which was filed on May 6, 2020 and is herein incorporated by reference.

### FIELD OF THE INVENTION

The invention relates to an apparatus for cleaning footwear and, more particularly but not exclusively, to sanitizing and/or disinfecting footwear.

### SUMMARY OF THE INVENTION

The invention relates to an apparatus for cleaning, sanitizing and/or disinfecting footwear. The apparatus comprises a set of brushes defining a footwear receiving space. A plurality of water carrying channels are provided for directing water into the footwear receiving space through said set of brushes. A soiled water receiving tank may be provided for collecting soiled water from the footwear receiving space. The apparatus is arranged such that relative movement of a piece of footwear received in the footwear receiving space with respect to the set of brushes in the presence of water being directed into the footwear receiving space via the plurality of water carrying channels cleans at least one surface of the received piece of footwear.

The summary of the invention does not necessarily disclose all the features essential for defining the invention; the invention may reside in a sub-combination of the disclosed features.

### BRIEF DESCRIPTION OF THE DRAWINGS

The foregoing and further features of the present invention will be apparent from the following description of preferred embodiments which are provided by way of example only in connection with the accompanying figures, of which:

FIG. 1 is a perspective view of a first embodiment of an apparatus in accordance with the invention;

FIG. 2 is an exposed perspective view of the apparatus of FIG. 1;

FIG. 3 is a perspective view of a second embodiment of an apparatus in accordance with the invention;

FIG. 4 is an enlarged reverse perspective view of the housing part of the apparatus of FIG. 3;

FIG. 5 is an exposed schematic view of a third embodiment of an apparatus in accordance with the invention;

FIG. 6 is a perspective view of a fourth embodiment of an apparatus in accordance with the invention;

FIG. 7 is an exposed perspective view of the apparatus of FIG. 6;

FIG. 8 is a perspective view of a fifth embodiment of an apparatus in accordance with the invention;

FIG. 9 is an exposed schematic view of a sixth embodiment of an apparatus in accordance with the invention;

FIG. 10 is a perspective view of a seventh embodiment of an apparatus in accordance with the invention;

FIG. 11 is an end view of an arrangement of water carrying channels for the apparatuses of the foregoing embodiments of the invention; and

FIG. 12 shows some additional features of the various embodiments of the apparatuses in accordance with the invention.

### DESCRIPTION OF PREFERRED EMBODIMENTS

The following description is of preferred embodiments by way of example only and without limitation to the combination of features necessary for carrying the invention into effect.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments, but not other embodiments.

The present description illustrates the principles of the present invention. It will thus be appreciated that those skilled in the art will be able to devise various arrangements that, although not explicitly described or shown herein, embody the principles of the invention and are included within its spirit and scope.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

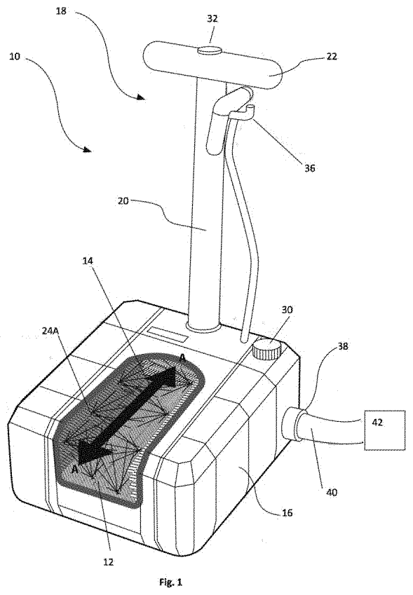

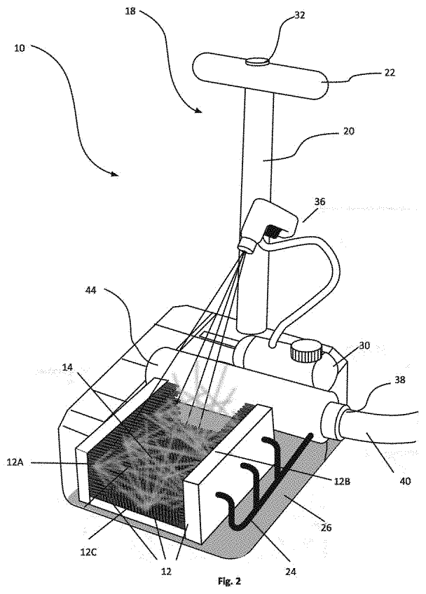

Referring to FIG. 1, shown is a perspective view of a first embodiment of an apparatus 10 in accordance with the invention. The apparatus 10 is intended for cleaning a piece of footwear using sprayed water whilst the piece of footwear is being worn by a user, but it will be understood that it is not essential to the purpose of the invention that a piece of footwear being cleaned in the apparatus 10 is being worn by a user whilst being cleaned. FIG. 2 shows an exposed perspective view of the apparatus of FIG. 1 to better understand the structure and function of the apparatus. The term “cleaning” as used herein may optionally comprise or include sanitizing and/or disinfecting as will be more fully described below.

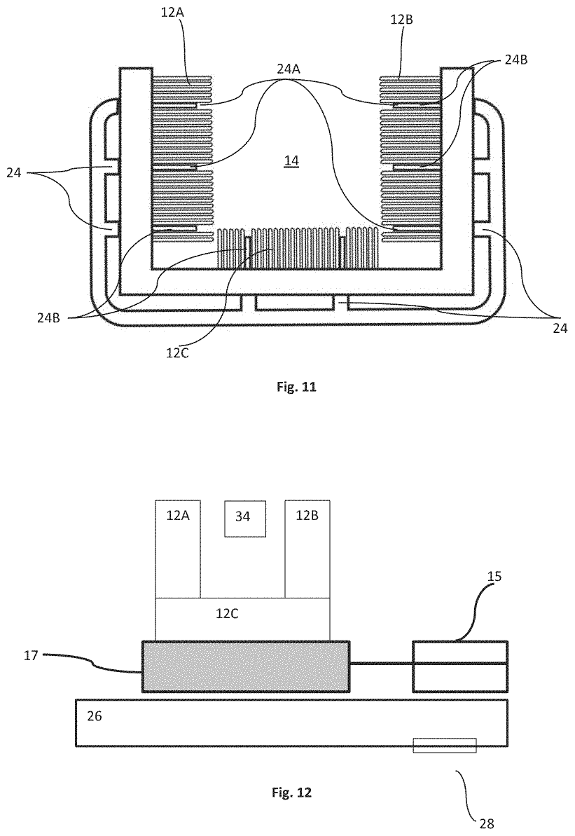

The apparatus 10 comprises a set of brushes 12 which between them define a footwear receiving space 14. The set of brushes 12 preferably comprises first and second laterally arranged inwardly facing brushes 12A, 12B, the inwardly facing surfaces of which define the footwear receiving space 14. More preferably, the set of brushes 12 includes a third base brush 12C whose upper facing surface defines a base surface of the footwear receiving space 14. The brushes 12A, 12B, 12C may be fixed in position with respect to a housing 16 of the apparatus. However, in some embodiments, the brushes 12A, 12B, 12C may be movably mounted within the housing 16 such that the brushes 12A, 12B, 12C can be caused to move reciprocally relative to the footwear receiving space 14. Reciprocal movement of the brushes 12A, 12B, 12C relative to the footwear receiving space 14 may be provided electrically or mechanically. In some embodiments, an electrically powered motor 15 (FIG. 12) may be provided to reciprocally move a cradle 17 on which the set of brushes 12 is mounted to thereby cause reciprocal movement of the set of brushes in a direction parallel with a longitudinal central axis A-A of the footwear receiving space 14.

It will be appreciated that the brushes 12A, 12B, 12C are selected to have bristles which, in use, are sufficiently stiff to tease dirt and other debris from one or more surfaces of a piece of footwear contacting said set of brushes 12 and yet not so stiff as to damage, e.g. scratch, the surfaces of the piece of footwear. The bristle stiffness of the brushes 12A, 12B, 12C may be selected dependent on a type of footwear intended to be cleaned in the apparatuses 10 in accordance with the invention although the intention is for the apparatuses 10 in accordance with the invention to clean a range of types of footwear. The set of brushes 12 may be manually replaceable or interchangeable with one or other sets of brushes.

Whilst in some embodiments of the apparatuses 10 of the invention, means 15, 17 may be provided to provide reciprocal movement of the brushes 12A, 12B, 12C relative to the footwear receiving space 14, in simpler embodiments of the apparatuses 10 of the invention, a user may manually move their foot within the footwear receiving space 14 to bring a piece of footwear they are wearing into contact with one or more of the brushes 12A, 12B, 12C. To assist a user in effecting movement of their foot within the footwear receiving space 14, the apparatus 10 may include a support device 18 which the user may grip when moving their foot. The support device 18 may be provided as a post 20 which depends upwardly at or near a rear edge of the housing 16. The post 20 may include a horizontally extending handle 22 for improved grip by a user.

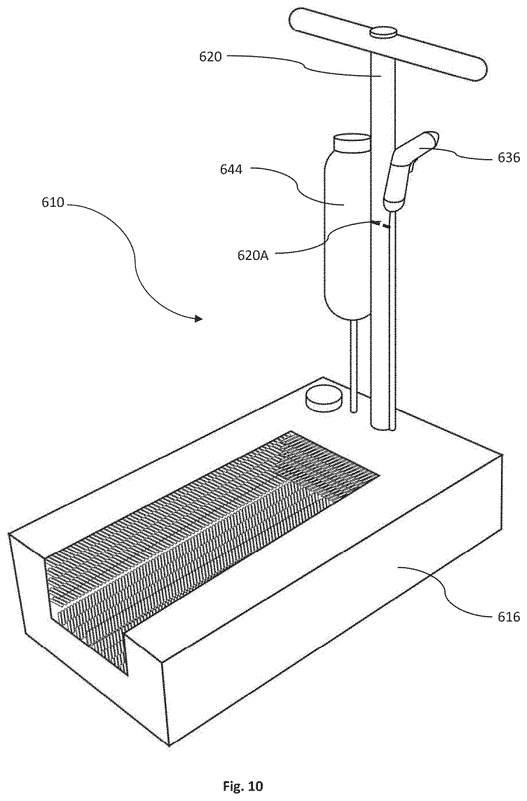

The apparatus 10 includes a plurality of water carrying channels 24 for directing water into the footwear receiving space 14 through said set of brushes 12. As can be better seen in at least FIGS. 2 and 10, the plurality of water carrying channels 24 are preferably arranged at similarly spaced intervals around three sides of the footwear receiving space 14.

It is preferred, but not essential, that each of the water carrying channels 24 extends through its respective brush 12 such that an outlet 24A of each water carrying channel 24 is positioned near to or adjacent a surface of the brush 12 facing the footwear receiving space 14. Furthermore, it is preferred that the outlet 24A of each water carrying channel 24 comprises a nozzle arranged to spray water under pressure into the footwear receiving space 14. In order to prevent damage to footwear being cleaned in the apparatus 10, it is preferred that a part 24B of each water carrying channel 24 which extends through or partially through its respective brush 12 comprises a flexible tube which flexes with movement of the bristles of the brush 12 when contacted by a piece of footwear.

The use of water under pressure in the form of sprays from the water carrying channel nozzles is preferred as this more readily assists in the loosening and removal of dirt and/or debris from surfaces of the piece of footwear being cleaned.

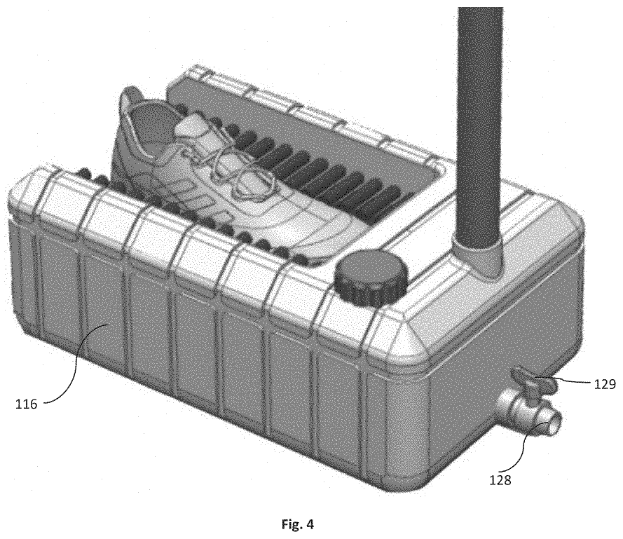

Preferably, the housing 16 provides a soiled water receiving tank 26 for collecting soiled water from the footwear receiving space 14. An outlet 28 (FIGS. 4 and 11) may be provided in a base of the soiled water receiving tank 26 to enable such soiled water to be conveniently disposed of.

The apparatus 10 preferably includes a tank 30 for receiving a sanitizer and/or a disinfectant solution. The tank 30 for receiving a sanitizer and/or a disinfectant solution is arranged such that the sanitizer and/or the disinfectant solution mixes slowly with the water being directed into the footwear receiving space 14 through the set of brushes 12 via the water carrying channels 24. This may be achieved by allowing a portion of the water to be directed into the footwear receiving space 14 to pass through the tank 30 for receiving a sanitizer and/or a disinfectant solution. Alternatively, it may be achieved by arranging for the tank 30 for receiving a sanitizer and/or a disinfectant solution to drip-feed sanitizer and/or disinfectant solution into a flow of the water to be directed into the footwear receiving space 14.

The apparatus 10 may include a manually operated actuator 32 such that, when a user places a piece of footwear into the footwear receiving space, manual operation of the actuator 32 causes water to be directed into the footwear receiving space 14 via the plurality of water carrying channels 24. Conveniently, the manually operated actuator 32 may be provided at the top of the post 20 or on the handle 22. Alternatively or additionally, the apparatus 10 may include an automatically operated actuator such that, when a user places a piece of footwear into the footwear receiving space 14, a detector 34 (FIG. 12) detects the presence of the piece of footwear in the footwear receiving space 14 and automatically triggers the actuator to cause water to be directed into the footwear receiving space 14 via the plurality of water carrying channels 24.

The apparatus 10 may include a hand-operated spray attachment 36 to enable a user of the apparatus 10 to clean an upper surface of a piece of footwear received in the footwear receiving space 14 and/or clean debris from the set of brushes 12.

In the embodiment of the apparatus 10 of FIGS. 1 and 2, the housing 16 has a connector 38 for connection to an external water supply to receive water for said plurality of water carrying channels 24. The connector 38 may comprise a hose fitting enabling the housing 16 to be connected via a hose 40 or the like to a faucet 42. In this embodiment, the apparatus 10 may include a water tank 44 for receiving water from the faucet 42, the water tank 44 preferably buffering the supply and flow of water to the water carrying channels 24.

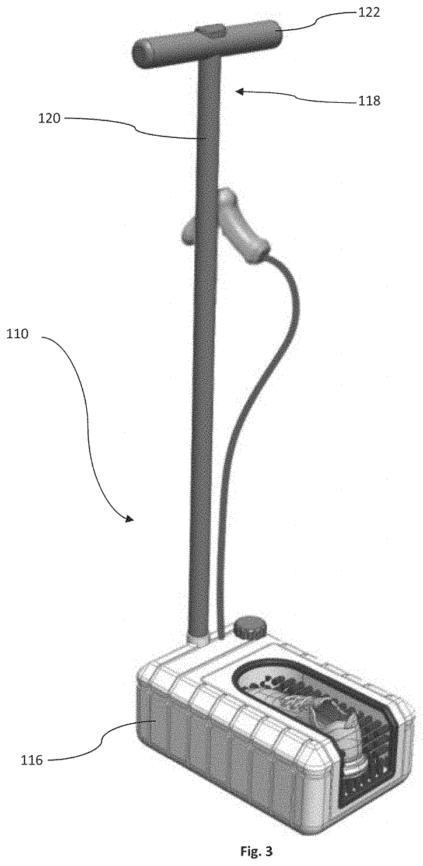

A second embodiment of an apparatus according to the invention is shown in FIGS. 3 and 4. In the description of this embodiment, like numerals, as used in the description of the first embodiment but preceded by the numeral “1”, will be used to denote like parts for reasons of brevity.

The apparatus 110 of this embodiment is largely of the same structure and configuration as that of the first embodiment, but differs from that of the first embodiment in that, as shown in FIG. 3, it has an elongated support device 118 comprising a post 120 and a handle 122 where the elongated support device 118 extends to a much greater height than the support device of the first embodiment. The support device 118 of this embodiment is ergonomically more convenient for use by elderly persons. The post 120 may be manually interchangeable with posts of other lengths.

FIG. 4 provides a reverse view of the housing 116 part of the apparatus 110 where the outlet 128 for draining the soiled water receiving tank (not shown) can be more readily seen. The outlet 128 may be opened and closed by means of a spigot 129.

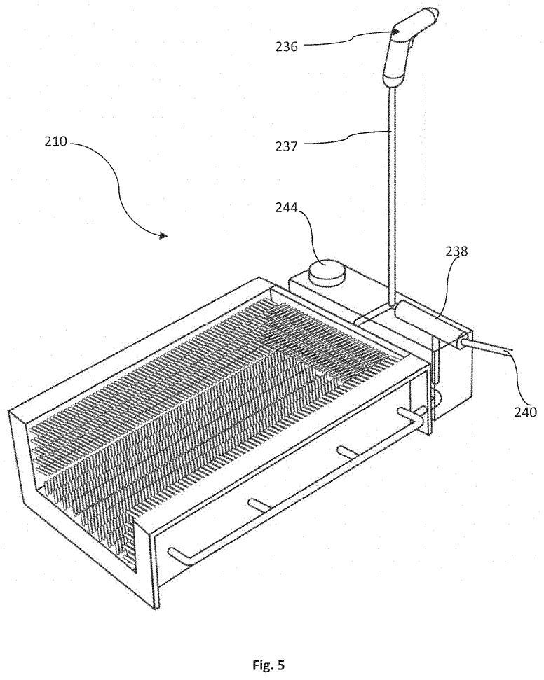

A third embodiment of an apparatus according to the invention is shown in schematic form in FIG. 5. In the description of this embodiment, like numerals, as used in the description of the first embodiment but preceded by the numeral “2”, will be used to denote like parts for reasons of brevity.

The apparatus 210 of this embodiment is largely of the same structure and configuration as that of the first embodiment, but differs from that of the first embodiment in that, as shown in FIG. 5, the hose connector 238, which connects to the hose 240, feeds water directly to both the water tank 244 and to a water line 237 of the hand-operated spray attachment 236. The hand-operated spray attachment 236 therefore operates under direct mains water pressure.

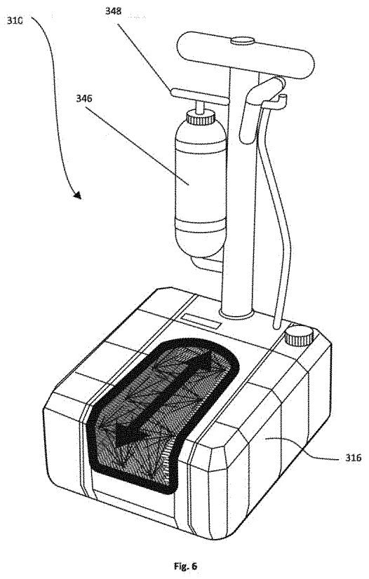

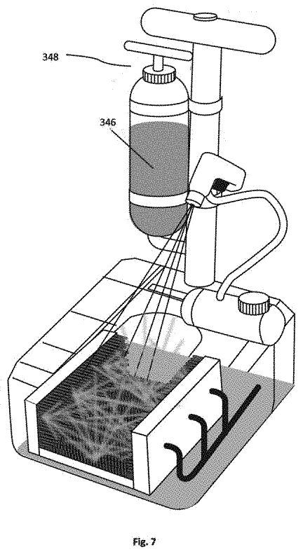

A fourth embodiment of an apparatus according to the invention is shown in FIGS. 6 and 7. In the description of this embodiment, like numerals as used in the description of the first embodiment but preceded by the numeral “3” will be used to denote like parts for reasons of brevity.

The apparatus 310 of this embodiment differs from that of the first embodiment in that it is not provisioned with any means for connecting to an external water supply but is provisioned with a water tank 346 for storing water for use in cleaning footwear. The water tank 346 is preferably able to be manually filled and to be able to be manually and/or automatically pressurized. As shown in FIGS. 6 and 7, it can be seen that the water tank 346 is provided with a manually operated pump 348 to pressurize said tank 346, although it will be understood that pressurization of the tank 346 may be provided by an electrically operated pump or the like. The advantage of the water tank 346 arrangement of this embodiment is that a user is not dependent on locating the apparatus 310 in reach of a water faucet.

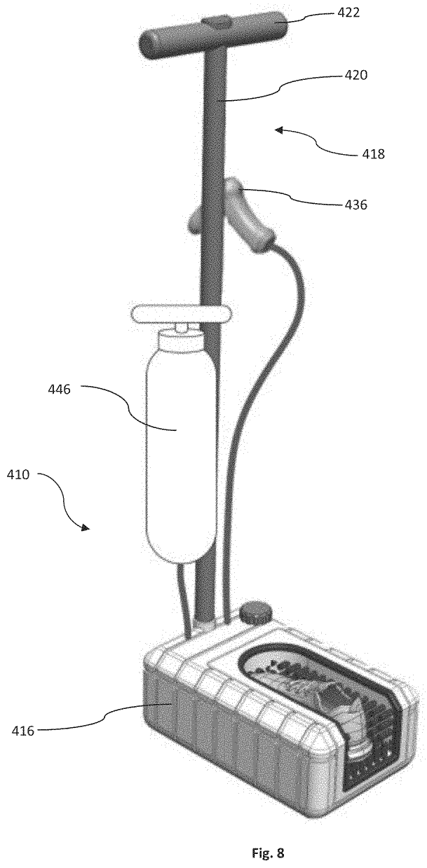

A fifth embodiment of an apparatus according to the invention is shown in schematic form in FIG. 8. In the description of this embodiment, like numerals, as used in the description of the first embodiment but preceded by the numeral “4”, will be used to denote like parts for reasons of brevity.

The apparatus 410 of this embodiment is largely of the same structure and configuration as that of the first and fourth embodiments, but differs from that of the fourth embodiment in that, as shown in FIG. 8, it has an elongated support device 418 comprising a post 420 and a handle 422 where the elongated support device 418 extends to a much greater height than the support devices of the first or fourth embodiments. Furthermore, in this embodiment, the water tank 446 for storing water for use in cleaning footwear is located on the post 520 at a position lower than a support for the hand-operated spray attachment 436.

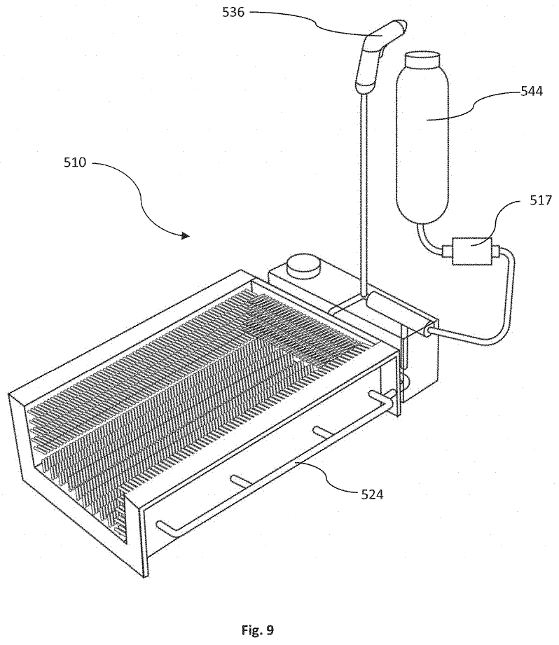

A sixth embodiment of an apparatus according to the invention is shown in schematic form in FIG. 9. In the description of this embodiment, like numerals, as used in the description of the first embodiment but preceded by the numeral “5”, will be used to denote like parts for reasons of brevity.

The apparatus 510 of this embodiment is largely of the same structure and configuration as that of the first and fourth embodiments, but differs from that of the fourth embodiment in that, as shown in FIG. 9, it has an electrically powered pump 517 for feeding water under pressure from the water tank 544 and to the water channels 524 and to the hand-operated spray attachment 536.

A seventh embodiment of an apparatus according to the invention is shown in schematic form in FIG. 10. In the description of this embodiment, like numerals, as used in the description of the first embodiment but preceded by the numeral “6”, will be used to denote like parts for reasons of brevity.

The apparatus 610 of this embodiment is largely of the same structure and configuration as that of the sixth embodiment, but differs from that of the sixth embodiment in that the electrically powered pump (not shown) for feeding water under pressure from the water tank 644 and to the water channels (not shown) and to the hand-operated spray attachment 636 is positioned within the housing 616. The housing 616 may also be elongated and/or also made deeper compared to other embodiments. The post 620 may be formed of two or more pieces as indicated by dashed line 620A to reduce the size of the retail shipment box for the product.

FIG. 11 an end view of an arrangement of water carrying channels 24 for the apparatuses 10, 110, 210, 310, 410, 510, 610 of the foregoing embodiments of the invention and shows more clearly the arrangement of the set of brushes 12 with respect to the water carrying channels 24 and the footwear receiving space 14. It also shows how the outlets 24A of the parts 24B of the water carrying channels 24 which extend through or partially through their respective brushes 12A,B,C are preferably recessed in the brushes 12A,B,C.

FIG. 12 illustrates some additional features of the various embodiments 10, 110, 210, 310, 410, 510, 610 of the apparatuses in accordance with the invention including the reciprocally moving cradle 17, the motor 15 for reciprocally moving the cradle 17, soiled water receiving tank 26 for collecting soiled water from the footwear receiving space 14, outlet 28 in the tank 26 to enable soiled water to be conveniently disposed of and the detector 34 for detecting the presence of a piece of footwear in the footwear receiving space 14. It will be appreciated that, where an apparatus in accordance with any of the embodiments of the invention is used indoors, it is preferable that the soiled water receiving tank 26 retains collected soiled water rather than allow it to drain from the tank 26 during use of the footwear apparatus of the invention. The outlet 28 enables retained soiled water in the tank 26 to be conveniently removed subsequent use of the footwear cleaning apparatus to clean some footwear. Alternatively, the tank 26 may be replaced by a slide-out tray whereby soiled water collected in the slide-out tray may be more easily disposed of and provide greater access to the inside of the housing of the footwear cleaning apparatus to clean said apparatus.

It is also preferred that the embodiments of the apparatus of the invention are provided with single holding tank for water to be mixed with sanitizing solution, eliminating the need for a mixing device and a second holding tank.

It will be seen from the drawings that many of the major constituent parts of the apparatuses 10, 110, 210, 310, 410, 510, 610 are accommodated with the housing 16, 116, 316, 416, 616 which acts to protect said parts as, in use, they are likely to be kicked by users if not covered by the housing 16, 116, 316, 416, 616.

While the invention has been illustrated and described in detail in the drawings and foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or of other embodiments not recited herein. Accordingly, the invention also provides embodiments that comprise combinations of one or more of the illustrative embodiments described above. Modifications and variations of the invention as herein set forth can be made without departing from the spirit and scope thereof, and, therefore, only such limitations should be imposed as are indicated by the appended claims.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art.

1. An apparatus for cleaning footwear comprising:
a set of brushes defining a footwear receiving space;
a plurality of water carrying channels for directing water into the footwear receiving space through said set of brushes;
wherein relative movement of a piece of footwear received in the footwear receiving space with respect to the set of brushes in the presence of water being directed into the footwear receiving space via the plurality of water carrying channels cleans at least one surface of the received piece of footwear.

2. The apparatus according to claim 1, wherein the apparatus includes a tank for receiving a sanitizer and/or a disinfectant solution, the arrangement being such that the sanitizer and/or the disinfectant solution mixes with the water being directed into the footwear receiving space through the set of brushes.

3. The apparatus according to claim 1, wherein the apparatus includes a manually operated actuator such that, when a user places a piece of footwear into the footwear receiving space, manual operation of the actuator causes water to be directed into the footwear receiving space via the plurality of water carrying channels.

4. The apparatus according to claim 1, wherein the apparatus includes an automatically operated actuator such that, when a user places a piece of footwear into the footwear receiving space, a detector detects the presence of the piece of footwear in the footwear receiving space and automatically triggers the actuator to cause water to be directed into the footwear receiving space via the plurality of water carrying channels.

5. The apparatus according to claim 1, wherein relative movement of a piece of footwear received in the footwear receiving space with respect to the set of brushes is provided by a user wearing the piece of footwear moving his foot with respect to the set of brushes or is provided by electrical or mechanical actuation of the brushes whereby the set of brushes are electrically or mechanically moved relative to the footwear receiving space.

6. The apparatus according to claim 5, wherein electrical or mechanical actuation of the brushes is provided by a motor which causes reciprocal movement of the brushes relative to the footwear receiving space.

7. The apparatus according to claim 1, wherein the set of brushes comprises two spaced apart laterally positioned brushes which define between them the footwear receiving space.

8. The apparatus according to claim 7, wherein the set of brushes includes a base brush which defines a base of the footwear receiving space.

9. The apparatus according to claim 1, wherein plurality of water carrying channels are arranged at similarly spaced intervals around three sides of the footwear receiving space.

10. The apparatus according to claim 1, wherein each of the plurality of water carrying channels extends through its respective brush such that an outlet of each water carrying channel is positioned near to or adjacent a surface of the brush facing the footwear receiving space.

11. The apparatus according to claim 10, wherein the outlet of each water carrying channel comprises a nozzle arranged to spray water under pressure into the footwear receiving space.

12. The apparatus according to claim 10, wherein a part of each water carrying channel which extend through its respective brush comprises a flexible tube which flexes with bristles of the brushes when contacted by a piece of footwear.

13. The apparatus according to claim 1, wherein the apparatus is connectable to an external water supply to receive water for said plurality of water carrying channels.

14. The apparatus according to claim 1, wherein the apparatus has a water tank for storing water for said plurality of water carrying channels.

15. The apparatus according to claim 14, wherein the water tank is a pressurized water tank.

16. The apparatus according to claim 15, wherein the pressurized water tank is pressurized manually using a manually operated pump forming part of the pressurized water tank or is pressurized automatically by an electrical pump.

17. The apparatus according to claim 1, wherein the apparatus includes a hand-operated spray attachment to enable a user of the apparatus to clean an upper surface of a piece of footwear received in the footwear receiving space and/or clean debris from the set of brushes.

18. The apparatus according to claim 1, wherein major portions of the water carrying channels for directing water into the footwear receiving space through said set of brushes are contained within a single housing.

19. The apparatus according to claim 18, wherein the housing provides the soiled water receiving tank.

20. The apparatus according to claim 1, wherein the apparatus provides a soiled water receiving tank for collecting soiled water from the footwear receiving space.
